(19) United States
(12) Patent Application Publication
(10) Pub. No.: US 2024/0294961 A1
(43) Pub. Date: Sep. 5, 2024

(54) Cellulose for Use in Cellulosic Ethanol-Producing Applications

(71) Applicant: SixRing Inc., Calgary (CA)

(72) Inventors: Alejandra ENRIQUEZ, Calgary (CA); Julie GREER, Calgary (CA); Markus WEISSENBERGER, Calgary (CA); Alexandria OSTASZEWSKI, Calgary (CA)

(21) Appl. No.: 18/460,258

(22) Filed: Sep. 1, 2023

(30) Foreign Application Priority Data
Mar. 3, 2023 (CA) 3191788

(51) Int. Cl. C12P 19/12; C12N 9/42

(52) U.S. Cl. CPC C12P 19/12; C12N 9/244; C12N 9/2445; C12Y 302/01006; C12Y 302/01021

(57) ABSTRACT

A process to hydrolyze cellulose into cellobiose comprises providing a reaction vessel and providing an inoculum of a bacteria or fungus capable of expressing one or more endo or exo-β-glucanase into reaction vessel. The bacterium or fungus is exposed to a source of cellulose having a kappa number of less than 10 and a hemicellulose content of less than 15% in an aqueous medium of pH between 5 and 9 at a temperature ranging from 20° C. to 40° C. for a period of time ranging from 1 to 30 days. The cellobiose is exposed to a bacterium or fungi or yeast, or combination which converts cellobiose to glucose or ethanol.

### FIELD OF THE INVENTION

The present invention is directed to the use of cellulose having a low Kappa number and hemicellulose content in order to generate substantial improvement in the conversion of cellulose to ethanol.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for biofuel production is starch which can yield its sugar much more readily than cellulose. This is due to the difference in structure as starch links glucose molecules together through alpha-1,4 linkages and cellulose links glucose with beta-1,4 linkages. The beta-1,4 linkages allow for crystallization of the cellulose, leading to a more rigid structure which is more difficult to break down.

The limitation that comes from solely concentrating the biofuel on extracting the sugars from starches prevents the utilization of the larger portion of biomass which comes in the form of lignocellulosic biomass (contains lignin, cellulose and hemicellulose) present in almost every plant on earth. A delignification reaction allows the recovery of cellulose from those lignocellulosic plants. Once the cellulose is separated from the other two biomass constituents i.e., lignin, and hemicellulose, further degradation of the cellulose generates cellobiose and/or glucose which can be further processed to bio ethanol.

Seen as a sustainable alternative to gasoline and with the goal of alleviating many countries' dependence on foreign oil, the biofuel industry is still hampered by its dependence on corn or sugar cane as their main sources of fuel, as they are both rich in starch. It is estimated that about a third of all corn production in the U.S. is directed to the ethanol fuel production. This is a situation which has disastrous consequences when the prices of gasoline go so low as to make corn-based biofuel unsustainable on a price viewpoint.

Across the world, many other large ethanol-producing countries, including China and Brazil, have shown some struggles in ethanol production from biomass as many companies are carrying large debts from the implementation of such processes and large plants have been to shut down or decrease production.

In Asia, palm oil prices have recently increased to their highest levels in years, which, in turn, will hamper the ability of Indonesia and Malaysia to produce local biofuel. Oil palm trunk, which is a valuable and plentiful resource in those countries, contains a large amount of starch which is more readily solubilized in water, compared to cellulose. Starch can then be heated and hydrolyzed to glucose by amylolytic enzymes without pre-treatment. However, the conventional oil palm trunk treatment requires high capital and operational costs and is therefore prohibitive to market entry. Moreover, the treatment carries a high probability of microbial contamination during starch processing.

In Europe, the biofuel industry (both biodiesel and bioethanol production) depends heavily on food-based feedstocks like virgin vegetable oils (i.e., rapeseed, palm oil, soy) for biodiesel and corn, wheat, and sugar beet for bioethanol. Simultaneously, concerns have been raised that making fuel out of crops displaces other crops and can inflate food prices. These concerns are leading to policy changes that incentivize a shift away from food-based biofuels.

To pivot from starches to cellulose for the production of glucose is preferable as it will provide near-unlimited amount of feedstock from waste biomass and reduce the competition with food source feedstock to generate glucose. However, the costs to do so are currently prohibitive. Cellulosic ethanol as it is called relies on the non-food part of a plant to be used to generate ethanol. This would allow the replacement of the current more widespread approach of making bioethanol by using corn or sugarcane. The diversity and abundance of these types of cellulose-rich plants would allow to maintain food resources mostly intact and capitalize on the waste generated from these food resources (such as cornstalk) to generate ethanol. Other cellulose sources such as grasses, algae and even trees fall under the cellulose-rich biomass which can be used in generating ethanol if a commercially viable process is developed.

The reason why starches are preferred to cellulose-rich sources to generate ethanol is that extraction of glucose from cellulose is substantially more difficult and resource intensive. To better understand the difference which raises this difficulty it is worthwhile pointing the similarities and differences between starch and cellulose.

Cellulose and starch are polymers which have the same repeat units of glucose. However, the differences between starch and cellulose can be seen in the way the repeating glucose monomers are connected to one another. In starch, the glucose monomers are oriented in the same direction. In cellulose, each successive glucose monomer is rotated 180 degrees in respect of the previous glucose monomer. This, in turn, ensures that the bonds between each monomeric glucose differs between starch and cellulose. In starch, the bonds (otherwise known as links) are referred to as α-1,4 linkages, in cellulose these bonds are referred to as β-1,4 linkages.

The difference between these bonds impacts the characteristics of starch and cellulose. Starch can dissolve in warm water while cellulose does not. Starch can be digested by humans, cellulose cannot. Starch is weaker than cellulose partly due to the fact that its structure is less crystalline than cellulose. Starch is, at its core, a method for plants to store energy, therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds (see FIG. 1). D-glucose is the building block of many polysaccharides, including cellulose. Glucose has two isomers: α-glucose (present in starches as branched polymers) and β-glucose (present in cellulose as repeating units of β-glucose subunits connected via a β-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed and, in turn, provide the structural strength needed to support plants.

### Hydrolysis of Cellulose

The hydrolysis of cellulose is the rate limiting step in the conversion of cellulose into bioethanol. The processes currently using cellulose as a starting material for bioethanol production require the conversion of cellulose into oligomers, then glucose, prior to the ultimate generation of ethanol. The fermentation of glucose using yeast is what leads to the production of ethanol. While that last step in biofuel production has been mastered for some time, the rate limiting step is the most crucial one and one which hinders a wider acceptance of bioethanol. The difficulty in overcoming this conversion of cellulose into glucose lies with the fact that cellulose has a crystalline structure which renders its conversion to glucose quite difficult because of the close packing of multiple cellulose polymers. This close packing imparts on cellulose it's inherent stability under a variety of chemical conditions. Cellulose polymers are generally insoluble in water, as well as a number of organic solvents. Cellulose is also generally insoluble when exposed to weak acids or bases.

In general, there are two main approaches to hydrolyze cellulose: chemical and enzymatic. The chemical method resorts to the use of concentrated strong acids to hydrolyze cellulose under conditions of high temperature and pressure. Many different types of acids, such as HCl and H₂SO₄, have been used in the past to achieve this. The use of one of these acids usually results in at least one of the following drawbacks: corrosion of the reaction vessel, difficulty of disposing of the discharged reactants, the cost of high energy intensive processes and others. The biofuel industry is generally reticent to use chemically hydrolyzed cellulose because of the presence of toxic by-products in the resulting glucose. These by-products, if introduced in the fermentation step, will negatively affect the delicate balance of the fermenting organism.

### Cost of Enzymatic Hydrolysis

It is known that the costs to extract biofuel from cellulose are higher than when doing so from starch. It is estimated that, on average, depending on location and availability of biomass, the cost for cellulose conversion is about 50% more that starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases). The generation of enzymes for enzymatic hydrolysis of cellulose is a time-consuming process and large volumes of enzyme are required to render the process commercially viable. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising endo-1,4-β-glucanases: exo-1,4-β-glucanases; and β-glucosidases) require extensive controls in place to maximize the reaction rates the enzymatic approach is expected to provide. Temperature, pH, salinity, concentration of substrate and product are all factors that may affect enzyme activity. Small deviations from the enzyme's optimal conditions will result in loss of function. The conversion of cellulose to glucose is done by a few different enzymes: endo-1,4-β-glucanases; exo-1,4-β-glucanases; and β-glucosidases, all of which have specific environmental conditions which must be met. These controls render the process cost prohibitive in some cases and/or limiting in their implementation.

PCT patent application WO9640970 (A1) discloses a method of producing sugars from materials containing cellulose and hemicellulose comprising: mixing the materials with a solution of about 25-90% acid by weight thereby at least partially decrystallizing the materials and forming a gel that includes solid material and a liquid portion; diluting said gel to an acid concentration of from about 20% to about 30% by weight and heating said gel to a temperature between about 80° C. and 100° C. thereby partially hydrolyzing the cellulose and hemicellulose contained in said materials; separating said liquid portion from said solid material, thereby obtaining a first liquid containing sugars and acid; mixing the separated solid material with a solution of about 25-90% acid until the acid concentration of the gel is between about 20-30% acid by weight and heating the mixture to a temperature between about 80° C. and 100° C. thereby further hydrolyzing cellulose and hemicellulose remaining in said separated solid material and forming a second solid material and a second liquid portion; separating said second liquid portion from said second solid material thereby obtaining a second liquid containing sugars and acid; combining the first and second liquids; and separating the sugars from the acid in the combined first and second liquids to produce a third liquid containing a total of at least about 15% sugar by weight, which is not more than 3% acid by weight.

In the paper entitled ‘Glucose production from cellulose through biological simultaneous enzyme production and saccharification using recombinant bacteria expressing the β-glucosidase gene’ by Ichikawa S. et al, (J Biosci Bioeng. 2019 March; 127(3):340-344), there is disclosed a cellulosic biomass saccharification technologies. Glucose was produced by the hydrolysis of 100 g/L Avicel cellulose for 10 days through biological simultaneous enzyme production and saccharification (BSES), and the product yield was similar to that obtained through BSES with purified β-glucosidase supplementation.

In the paper entitled ‘A novel facile two-step method for producing glucose from cellulose’ (Bioresource Technology Volume 137, June 2013, Pages 106-110) a two-step acid-catalyzed hydrolysis methodology is disclosed where cellulose is hydrolyzed to glucose with high yield and selectivity under mild conditions. Its approach involves a multi-step hydrolysis, comprising as first step, the depolymerization of microcrystalline cellulose in phosphoric acid to cellulose oligomer at 50° C. The second step involves the precipitation of the oligomer by ethanol and subsequent hydrolysis with dilute sulfuric acid.

The review article “BIODIVERSITY OF CELLULASE PRODUCING BACTERIA AND THEIR APPLICATIONS” by Akhtar et al. (2014) reports that many microorganisms are able to produce and secrete cellulolytic, hemicellulolytic and lignolytic enzymes. These microorganisms are found among extremely variegated taxonomic groups inhabiting diverse habitats from extreme thermophillic conditions to polar regions and aerobic to anaerobic systems. Already by 1976, an impressive collection of more than 14,000 fungi active against cellulose and other insoluble fibres were reported. Most of the cellulolytic bacteria fall within the phyla Actinobacteria, Bacteroidetes, Fibrobacteres, Firmicutes and Proteobacteria. Phyla Actinobacteria and Firmicutes constitute 80% of the isolated cellulose degrading bacteria.

In the paper “Cleaning carbohydrate impurities from lignin using Pseudomonas fluorescens” Ghosh et al. (2019), it was demonstrated that lignin biomass could be exposed to Pseudomonas fluorescens (P. fluorescens) to selectively biodegrade cellulose and hemicellulose therefrom. P. fluorescens is a non-pathogenic bacterium capable of producing cellulolytic enzymes and is adapted to cleave polymeric carbohydrates into free sugars as use them as their carbon source. This paper concludes that P. fluorescens is yet another organism which can be used to convert lignocellulosic biomass into free sugars.

In the paper “Direct ethanol production from cellulose by consortium of Trichoderma reesei and Candida molischiana” (2019). Bu et al. disclose the use of Trichoderma reesei and Candida molischiana to generate ethanol from cellulose. It is stated that the cellulose was hydrolyzed through an enzymatic saccharification process using Trichoderma reesei cellulases. The resulting sugar was then utilized by Candida molischiana to generate ethanol.

In the paper entitled ‘Dilute-acid Hydrolysis of Cellulose to Glucose from Sugarcane Bagasse’ from Dussan et al. (CHEMICAL ENGINEERING TRANSACTIONS VOL. 38, 2014), there is disclosed a method of generating ethanol through the hydrolysis of cellulose. Sugarcane bagasse is used as a substrate for ethanol production, optimum conditions for acid hydrolysis of cellulose fraction were assessed. The glucose thus generated was fermented to ethanol using the yeast (Scheffersomyces stipitis).

The hydrolysis of cellulose is, as seen from the above, limited by the structure of cellulose itself but also by the approaches taken to degrade it into a biofuel. The production of a robust, low-cost process from cellulose has not yet been achieved.

U.S. Pat. No. 9,663,807 discloses the preparation of ethanol by using lignocellulosic biomass such as corn stover which is pre-treated to remove C5 compounds (derived from hemicellulose), to leave C6 solids to be subsequently subjected to a simultaneous saccharification and fermentation (SSF) process. It was noted that simultaneous saccharification and fermentation could be performed at temperatures suitable for ethanol production by the yeast (e.g., about 37° C.) but this, in turn, was less than optimal for the cellulase enzyme. Consequently, the yields from such enzymes were lower because their activity was impeded by the presence of lignin on which cellulase enzymes could bind. In that case, it was discovered that addition of a lignin-binding agent, such as clarified thin stillage and/or Anaerobic Membrane Bioreactor (AnMBR) effluent could result in increased glucose yield during enzyme hydrolysis.

In light of the above, there is a profound need to develop a process for biofuel generation from waste biomass as an abundant and untapped source of renewable biofuels that does not compete with a food source, such as corn. In that respect, a lignocellulosic biomass from which cellulose is extracted is much more highly attractive as it will leave food sources available to fulfill their primary intended purpose and yet still generate a substantial cellulosic yield. The aforementioned is also substantiated with the tremendous efforts to convert waste biomass to biofuels using different approaches which have almost all failed to achieve this goal for subsequent conversion to glucose and ultimately, ethanol.

The inventors have surprisingly and unexpectedly found that the characteristics of the cellulose obtained from a specific type of delignification approach have a substantial impact on the downstream hydrolysis of said cellulose. A cellulose having a low lignin content provides an advantageous substrate for the production of glucose. More preferably, a cellulose having a low lignin and low hemicellulose content provides a substrate which can be even more easily converted to glucose. Subsequent conversion of glucose to ethanol results in substantial savings in the production of the latter and an increased usage of cellulose to make ethanol.

### SUMMARY OF THE INVENTION

According to another aspect of the present invention, there is provided a process to hydrolyze cellulose into cellobiose, said process comprising the following steps:
providing a reaction vessel;
providing a source of cellulose into said reaction vessel: wherein said source of cellulose having has a kappa number of less than 10, preferably the kappa number is 5 or less, more preferably the kappa number is 2 or less; and a hemicellulose content of less than 15%; preferably less than 10%, even more preferably less than 5% w/w of the total weight of the source of cellulose;
providing an organism capable of expressing one or more β-glucanases inoculum into said reaction vessel;
exposing said an organism capable of expressing one or more β-glucanases to said source of cellulose in an aqueous medium; and
optionally, recovering the supernatant comprising cellobiose.

According to a preferred embodiment of the present invention, the process further comprises a step of exposing the cell supernatant to an organism to convert cellobiose to glucose, wherein said organism selected from the group consisting of: a bacterium, a fungus, a yeast, and a combination thereof, such as Aspergillus brasiliensis, Trichoderma reesei and Pseudomonas protegens. Preferably, said organism capable of expressing one or more β-glucanase is selected from the group consisting of bacteria and fungi capable of producing endo-β-glucanase enzymes or exo-β-glucanase enzymes. Also preferably, said organism capable of expressing one or more β-glucanase is a bacterium of the phylum Bacteroidetes. Also preferably, said organism capable of expressing one or more β-glucanase is Cytophaga hutchinsonii.

According to a preferred embodiment of the present invention, said organism capable of expressing one or more β-glucanase is a bacterium of the phylum Proteobacteria. Preferably, said organism capable of expressing one or more β-glucanases is Pseudomonas protegens.

According to a preferred embodiment of the present invention, the resulting glucose is exposed to one of Zymomonas mobilis or yeast Saccharomyces cerevisiae in a step to convert the glucose to ethanol.

According to a preferred embodiment of the present invention, the organism used to convert cellobiose to glucose is a bacteria or fungi capable of producing β-glucosidase enzymes.

According to a preferred embodiment of the present invention, the organism used to convert cellobiose to glucose is a fungus of the phylum Ascomycota.

According to a preferred embodiment of the present invention, the organism used to convert cellobiose to glucose is a bacterium of the phylum Proteobacteria.

Preferably, said source of cellulose is exposed to an organism at a temperature between 20° C. to 40° C. Preferably, said organism is incubated with said source of cellulose for a period of time ranging from 1 to 30 days. Preferably, said aqueous medium has a pH of about 5.0 to 8.0.

According to another aspect of the present invention, there is provided a process to hydrolyze cellulose into glucose, said process comprising the following steps:
providing a reaction vessel;
providing a source of cellulose into said reaction vessel; wherein said source of cellulose having has a kappa number of less than 10, preferably the kappa number is 5 or less, more preferably the kappa number is 2 or less; and a hemicellulose content of less than 15%; preferably less than 10% w/w; even more preferably less than 5% w/w of the total weight of the source of cellulose;
providing an inoculum of an organism capable of expressing one or more β-glucanases and β-glucosidase into said reaction vessel;
exposing said an organism capable of expressing one or more β-glucanases and β-glucosidase to said source of cellulose in an aqueous medium; and
optionally, recovering the supernatant comprising glucose.

Preferably the process employs a bacteria or fungi capable of producing said organism capable of expressing endo- or exo-β-glucanase and β-glucosidase. Preferably, said bacteria or fungi capable of producing said organism capable of expressing endo- or exo-β-glucanase and β-glucosidase is selected from the group consisting of: Ascomycota, Proteobacteria, and combinations thereof. Preferably, said organism capable of expressing one or more β-glucanases and β-glucosidase is Trichoderma reesei. Also preferably, said an organism capable of expressing one or more β-glucanases and β-glucosidase is Pseudomonas protegens.

According to a preferred embodiment of the present invention, said cellulose source is exposed to an organism at a temperature between 30° C. to 40° C.

According to a preferred embodiment of the present invention, said organism is incubated with said cellulose source for a period of time ranging from 1 to 30 days. Preferably, said aqueous medium has a pH of about 5.0 to 9.0.

According to an aspect of the present invention, there is provided a process to convert cellulose to cellobiose (and optionally, to glucose or ethanol), said process comprising the steps of:
providing a reaction vessel;
providing a source of cellulose into said reaction vessel: wherein said source of cellulose having a content of hemicellulose of less than 15%, preferably less than 10% and more preferably less than 5%, and a kappa number of less than 10, more preferably less than 5, and even more preferably, less than 2;
providing an organism capable of expressing one or more β-glucanases into said reaction vessel;
exposing said organism to said source of cellulose in an aqueous medium of pH between 5 and 9 at a temperature ranging from 30° C. to 35° C. for a period of time ranging from 1 to 30 days;
optionally, recovering the supernatant comprising cellobiose;
optionally, exposing said supernatant comprising cellobiose to a bacteria or fungi that produces β-glucosidase, for the conversion of cellobiose to glucose;
optionally, recovering the supernatant comprising glucose; and
optionally, exposing said supernatant comprising glucose to an ethanologenic organism for the fermentation glucose to ethanol.

Preferably, the temperature inside the reaction vessel during said exposure time does not exceed 70° C.: more preferably 60° C., and even more preferably, 50° C.

According to another aspect of the present invention, there is provided a process to convert cellulose to cellobiose (and optionally, to glucose or ethanol), said process comprising the steps of:
providing a reaction vessel;
providing a source of cellulose into said reaction vessel; wherein said source of cellulose having a content of hemicellulose of less than 15%, preferably less than 10% and more preferably less than 5%, and a kappa number of less than 10, more preferably less than 5, and even more preferably, less than 2;
providing a Cytophaga hutchinsonii inoculum into said reaction vessel;
exposing said a Cytophaga hutchinsonii to said source of cellulose in an aqueous medium of pH between 5 and 9 at a temperature ranging from 20° C. to 40° C. for a period of time ranging from 0 to 30 days;
optionally, recovering the supernatant comprising cellobiose;
optionally, exposing said supernatant comprising cellobiose to a bacteria or fungi that produces β-glucosidase for the conversion of cellobiose to glucose;
optionally, recovering the supernatant comprising glucose; and
optionally, exposing said supernatant comprising glucose to an ethanologenic organism for the fermentation glucose to ethanol.

According to another aspect of the present invention, there is provided a process to convert cellulose to cellobiose (and optionally, to glucose and/or ethanol), said process comprising the steps of:
providing a reaction vessel;
providing a source of cellulose into said reaction vessel; wherein said source of cellulose having a content of hemicellulose of less than 15%, preferably less than 10% and more preferably less than 5%, and a kappa number of less than 10, more preferably less than 5, and even more preferably, less than 2;
providing a Pseudomonas protegens inoculum into said reaction vessel;
exposing said a Pseudomonas protegens to said source of cellulose in an aqueous medium of pH between 5 and 9 for a period of time ranging from 0 to 30 days;
optionally, recovering the supernatant comprising cellobiose;
optionally, exposing said supernatant comprising cellobiose to a bacteria or fungi that produces β-glucosidase for the conversion of cellobiose to glucose;
optionally, recovering the supernatant comprising glucose; and
optionally, exposing said supernatant comprising glucose to an ethanologenic organism for the fermentation glucose to ethanol.

Preferably, the temperature inside the reaction vessel during said exposure time does not exceed 70° C.: more preferably 60° C., and even more preferably, 50° C.

According to another aspect of the present invention, there is provided a process to convert cellulose to glucose (and optionally, to ethanol), said process comprising the steps of:
providing a reaction vessel;
providing a source of cellulose into said reaction vessel: wherein said source of cellulose having a content of hemicellulose of less than 15%, preferably less than 10% and more preferably less than 5%, and a kappa number of less than 10, more preferably less than 5, and even more preferably, less than 2;
providing a fungal or bacterial inoculum, preferably Trichoderma reesei into said reaction vessel;
exposing said a Trichoderma reesei, to said source of cellulose in an aqueous medium of pH between 5 and 9 at 200 rpm for a period of time ranging from 0 to 30 days;
optionally, recovering the supernatant comprising glucose; and
optionally, exposing said supernatant comprising glucose to an ethanologenic organism for the fermentation of glucose to ethanol.

Preferably, the temperature inside the reaction vessel during said exposure time does not exceed 70° C.: more preferably 60° C., and even more preferably, 50° C.

According to a preferred embodiment of the present invention, the method of delignification of biomass material which yields a low lignin and low hemicellulose cellulose also referred to as low kappa number and low hemicellulose content cellulose (also referred to as modified Caro's acid delignified (MCA delignified) cellulose) used in the cellulose to cellobiose (and ultimately, glucose and ethanol) conversion experiments comprise:
the source of cellulose is a lignocellulosic biomass delignified by exposure to a modified Caro's acid composition selected from the group consisting of: composition A; composition B and Composition C;
wherein said composition A comprises:
   sulfuric acid in an amount ranging from 20 to 70 wt % of the total weight of the composition;
   a modifier component comprising an amine moiety and a sulfonic acid moiety selected from the group consisting of: taurine; taurine derivatives; and taurine-related compounds; and
   a peroxide;
wherein said composition B comprises:
   an alkylsulfonic acid; and
   a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
   sulfuric acid;
   a two-part modifier component comprising:
      a compound comprising an amine moiety; and
      a compound comprising a sulfonic acid moiety; and
   a peroxide;
      for a period of time sufficient to remove substantially all of the lignin present on said biomass material. To alleviate the text, the above-described process can be referred to hereinafter as the modified Caro's acid delignification process, as well as the obtained cellulose can be referred to as modified Caro's acid delignified cellulose or “MCA cellulose” to indicate the method of delignification employed to obtain said cellulose.

Preferably, said sulfuric acid, said compound comprising an amine moiety and a sulfonic acid moiety and said peroxide are present in a molar ratio of no more than 15:1:1. Also preferably, said sulfuric acid and said compound comprising an amine moiety and a sulfonic acid moiety are present in a molar ratio of no less than 3:1.

According to a preferred embodiment of the approach to obtain low lignin cellulose, said delignification lasts from 2 to 20 hours.

According to a preferred embodiment of the approach to obtain low lignin cellulose, said delignification is carried out at temperatures below 50° C. Preferably, the delignification is carried out at temperatures below 40° C.

According to a preferred embodiment of the present invention, the process described herein that generates cellobiose (or glucose) from cellulose, employs a cellulose with low kappa number and low hemicellulose content, where said cellulose has the following characteristics: particle size ranging from 0-1000 microns, a content of hemicellulose of less than 15%, preferably less than 10%; more preferably less than 5% w/w of the total weight of the cellulose; and a kappa number of less than 10, more preferably less than 5, and even more preferably, less than 2.

According to another aspect of the present invention, there is provided a use of a source of cellulose having a kappa number of less than 10, preferably the kappa number is 5 or less, more preferably the kappa number is 2 or less; and a hemicellulose content of less than 15%; preferably less than 10% w/w, even more preferably, less than 5% w/w of the total weight of the source of cellulose; in a process to hydrolyze cellulose into cellobiose, wherein said process comprising the following steps:
providing a reaction vessel;
providing said source of cellulose into said reaction vessel;
providing an organism capable of expressing one or more β-glucanases into said reaction vessel;
exposing said an organism capable of expressing one or more β-glucanases to said source of cellulose in an aqueous medium; and
optionally, recovering the supernatant comprising cellobiose.

According to another aspect of the present invention, there is provided a use of a source of cellulose having a kappa number of less than 10, preferably the kappa number is 5 or less, more preferably the kappa number is 2 or less; and a hemicellulose content of less than 15%; preferably less than 10% w/w, even more preferably, less than 5% w/w of the total weight of the source of cellulose; in a process to hydrolyze cellulose into cellobiose (and optionally, to glucose or ethanol), wherein said process comprising the following steps:
providing a reaction vessel;
providing said source of cellulose into said reaction vessel;
providing an inoculum of an organism capable of expressing one or more β-glucanases and β-glucosidase into said reaction vessel;
exposing said an organism capable of expressing one or more β-glucanases and β-glucosidase to said source of cellulose in an aqueous medium;
optionally, recovering the supernatant comprising cellobiose;
optionally, exposing said supernatant comprising cellobiose to a bacteria or fungi that produces β-glucosidase for the conversion of cellobiose to glucose;
optionally, recovering the supernatant comprising glucose; and
optionally, exposing said supernatant comprising glucose to an ethanologenic bacteria or fungi for the fermentation glucose to ethanol.

According to another aspect of the present invention, there is provided a process wherein said cellulose is characterized by an absence of prior exposure to bleaching chemicals selected from the group consisting of: sodium hydrosulphite (Na₂S₂O₄); pentasodium salt diethylenetriaminepentaacetic acid; amine borane (CH₃)₃CNH₂—BH₃; borane ammonia complex BH₃—NH₃; sodium percarbonate; formamidine sulphinic acid; sodium perborate; and chlorine dioxide. The person skilled in the art will understand that, in the context of the present application, where there is a reference to bleaching of pulp, it is to be understood that the bleaching refers to a separate and distinct step of pulp processing. Consequently, the pulp used according to a preferred process of the present invention, is intended on being a pulp which has not undergone a separate bleaching step post-delignification. Such a treatment step is understood to not be economically viable when the ultimate goal of the cellulose is to be used to generate ethanol.

### BRIEF DESCRIPTION OF THE FIGURES

Features and advantages of embodiments of the present application will become apparent from the following detailed description and the appended figures, in which:

FIG. 1 is a depiction of glucose monomers present in a cellulose polymer and their β-1,4-linkages, demonstrating the binding sites of β-glucanase and β-glucosidase enzymes;

FIG. 2 illustrates a graphical representation of the β-glucanase enzymatic activity of various microorganisms exposed to a modified Caro's acid delignified cellulose; and

FIG. 3 illustrates a graphical representation of the β-glucosidase enzymatic activity of various microorganisms exposed to a modified Caro's acid delignified cellulose and cellobiose,

### DETAILED DESCRIPTION OF THE INVENTION

The description that follows, and the embodiments described therein, is provided by way of illustration of an example, or examples, of particular embodiments of the principles of the present invention. These examples are provided for the purposes of explanation, and not limitation, of those principles and of the invention.

According to a preferred embodiment of the present invention, Cytophaga hutchinsonii. Pseudomonas protegens and Trichoderma reesei are used to hydrolyze a modified Caro's acid delignified (MCA delignified) cellulose which has a kappa number ranging between 0-2.

According to a preferred embodiment of the present invention, the process to hydrolyze cellulose into cellobiose comprises the following steps:
providing a reaction vessel;
providing an organism capable of expressing one or more β-glucanases into said vessel;
exposing said organism to a source of cellulose having a kappa number of less than 10, more preferably less than 5 and even more preferably, less than 2, in an aqueous medium.

According to a preferred embodiment of the present invention, said organism capable of expressing one or more β-glucanases is a bacterium or fungus.

Preferably, the bacterium is a member of the phylum Bacteroidetes or of the phylum Proteobacteria. More preferably, the bacterium is Cytophaga hutchinsonii. or Pseudomonas protegens.

Preferably, the fungus is a member of the phylum Ascomycota. More preferably, the fungus is Trichoderma reesei.

According to a preferred embodiment of the present invention, the process of exposing said cellulose source to said organism occurs at a temperature of less than 40° C. Preferably said process occurs at a temperature between 25° C. to 37° C.

According to a preferred embodiment of the present invention, the process of exposing said cellulose source to said organism occurs for a period of time ranging from 1 to 60 days, preferably between 3 and 30 days.

According to a preferred embodiment of the present invention, said aqueous medium has a pH of about 5.0 to 8.0. Preferably, said aqueous medium is maintained at a pH of 6.0-7.5.

Preferably, the process further comprises a step of exposing the cell supernatant to an ethanologenic organism which converts cellobiose to glucose or ethanol.

According to a preferred embodiment of the present invention, said ethanologenic organism is a bacterium or a fungi. More preferably, said ethanologenic organism is Saccharomyces cerevisiae.

Internally generated data has shown that the modified Caro's acid delignified (MCA delignified) cellulose with a lower kappa number have generated higher enzyme activity in the first exposure to the organism that produces endo and exo-β-glucanases. This first exposure is meant to hydrolyze the β-1,4-glycosidic bonds of the cellulose to generate oligosaccharides and therefore generate the bulk of the glucose precursor material (in this case, cellobiose). It is understood that the second step of the process according to a preferred embodiment of the present invention is straightforward as it does not deviate from the common approach of cellobiose conversion to glucose. It is established that the first step is more determinant of the extent of biomass conversion to glucose since it will generate as main primary product, the cellobiose, which is subsequently employed in conversion to glucose. It is desirable that the cellulose degradation to cellobiose be maximized at this step, otherwise it will prevent the second step of the process from having a significant impact.

### Process to Obtain Modified Caro's Acid Delignified Cellulose

According to a preferred embodiment of the present invention, the method of delignification of biomass material which yields a modified Caro's acid delignified cellulose (also referred to as MCA cellulose) used in the cellulose to cellobiose (and ultimately, glucose) conversion experiments comprise:
providing a biomass material comprising cellulose fibers and lignin;
exposing said biomass material requiring delignification to a modified Caro's acid composition selected from the group consisting of: composition A; composition B and Composition C;
   wherein said composition A comprises:
sulfuric acid in an amount ranging from 20 to 70 wt % of the total weight of the composition;
a modifier component comprising an amine moiety and a sulfonic acid moiety selected from the group consisting of: taurine; taurine derivatives; and taurine-related compounds; and
a peroxide;
   wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;
   wherein said composition C comprises:
sulfuric acid;
a two-part modifier component comprising:
   a compound comprising an amine moiety; and
   a compound comprising a sulfonic acid moiety; and
a peroxide;
   for a period of time sufficient to remove substantially all of the lignin present on said biomass material. The process can be carried out for a varying duration of time depending on the particle size of the biomass being fed into the process. The process can last from 2 to 20 hours depending on that characteristic. Moreover, the temperature of the resulting mixture also has an impact on the duration of the process as the reaction is highly exothermic, precautions are taken to prevent a runaway degradation of the cellulose. This would result in a carbon black resulting product with no value. The process is preferably run at temperatures below 50° C., more preferably at temperatures below 40° C. The process of delignification is preferably performed with a cooling means adapted to control the heat generated by the chemical reaction of delignification and maintain the temperature to avoid an undesirable ‘runaway’ reaction.

Preferably, said sulfuric acid, said compound comprising an amine moiety and a sulfonic acid moiety and said peroxide are present in a molar ratio of no more than 15:1:1.

According to a preferred embodiment of the approach to obtain low lignin cellulose, said sulfuric acid and said compound comprising an amine moiety and a sulfonic acid moiety are present in a molar ratio of no less than 3:1.

Preferably, said modifier component comprising an amine moiety and a sulfonic acid moiety is selected from the group consisting of: taurine; taurine derivatives; and taurine-related compounds.

According to a preferred embodiment of the approach to obtain low lignin cellulose, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl. Preferably, said linear alkylaminosulfonic acid is selected form the group consisting of: methyl; ethyl (taurine); propyl; and butyl. Preferably, branched aminoalkylsulfonic acid is selected from the group consisting of: isopropyl; isobutyl; and isopentyl.

According to a preferred embodiment of the approach to obtain low lignin cellulose, said compound comprising an amine moiety and a sulfonic acid moiety is taurine.

According to a preferred embodiment of the approach to obtain low lignin cellulose, said sulfuric acid and compound comprising an amine moiety and a sulfonic acid moiety are present in a molar ratio of no less than 3:1.

According to a preferred embodiment of the approach to obtain low lignin cellulose, said compound comprising an amine moiety is an alkanolamine is selected from the group consisting of: monoethanolamine; diethanolamine; triethanolamine; and combinations thereof.

According to a preferred embodiment of the approach to obtain low lignin cellulose, said compound comprising a sulfonic acid moiety is selected from the group consisting of: alkylsulfonic acids; arylsulfonic acids; and combinations thereof.

Preferably, said alkylsulfonic acid is selected from the group consisting of: alkylsulfonic acids where the alkyl groups range from C1-C6 and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Preferably, said arylsulfonic acid is selected from the group consisting of: toluenesulfonic acid; benzesulfonic acid; and combinations thereof.

According to a preferred embodiment of the approach to obtain low lignin cellulose, said alkylsulfonic acid; and said peroxide are present in a molar ratio of no less than 1:1.

Preferably, said compound comprising a sulfonic acid moiety is methanesulfonic acid.

According to a preferred embodiment of the approach to obtain low lignin cellulose, said Composition C may further comprise a compound comprising an amine moiety. Preferably, the compound comprising an amine moiety has a molecular weight below 300 g/mol. Preferably also, the compound comprising an amine moiety is a primary amine. More preferably, the compound comprising an amine moiety is an alkanolamine. Preferably, the compound comprising an amine moiety is a tertiary amine. According to a preferred embodiment of the approach to obtain low lignin cellulose, the alkanolamine is selected from the group consisting of: monoethanolamine; diethanolamine; triethanolamine; and combinations thereof. Preferably, the alkanolamine is triethanolamine.

According to a preferred embodiment of the approach to obtain low lignin cellulose, said in Composition C, said sulfuric acid and said a compound comprising an amine moiety and said compound comprising a sulfonic acid moiety are present in a molar ratio of no less than 1:1:1.

Preferably, in Composition C, said sulfuric acid, said compound comprising an amine moiety and said compound comprising a sulfonic acid moiety are present in a molar ratio ranging from 28:1:1 to 2:1:1.

Preferably, in Composition C, said compound comprising an amine moiety is triethanolamine and said compound comprising a sulfonic acid moiety is methanesulfonic acid.

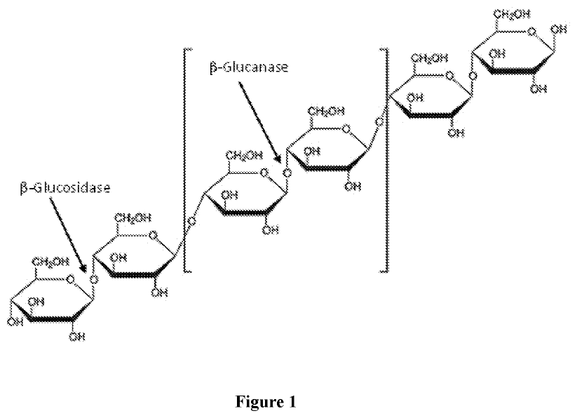

It is known by those skilled in the art that the biodegradation of cellulose utilizes two different enzymes comprising endo-1,4-β-glucanase or exo-1,4-β-glucanase; and β-glucosidase. The 1,4-β-glucanase enzymes hydrolyze the glycosidic bonds between the glucose monomers within the cellulose chain. The β-glucosidase enzymes catalyze the hydrolysis of the glycosidic bonds of cellobiose, or of the glucose monomers at the ends of the cellulose chain. The different locations on cellulose in which these two enzymes will interact is depicted in FIG. 1. Alone, β-glucanase will generate cellobiose, but working in tandem with β-glucosidase, glucose will be produced.

A known microorganism capable of converting cellulose to cellobiose was used as a control in the comparison of various bacteria and fungi on biodegradability of MCA delignified cellulose. Four additional microorganisms, encompassing bacteria and fungi, were tested to measure cellulase enzyme activity (Table 1). The organisms were grown within a temperature range of 25-40° C., a pH range of 5-9 and shaking at 150 rpm. The media and growth conditions of each microorganism was designed to be optimal for each respectively. MCA cellulose was added at a 1% w/w loading of the total volume of the cultures. Cultures were also all spiked with 0.5% cellobiose to induce β-glucosidase activity.

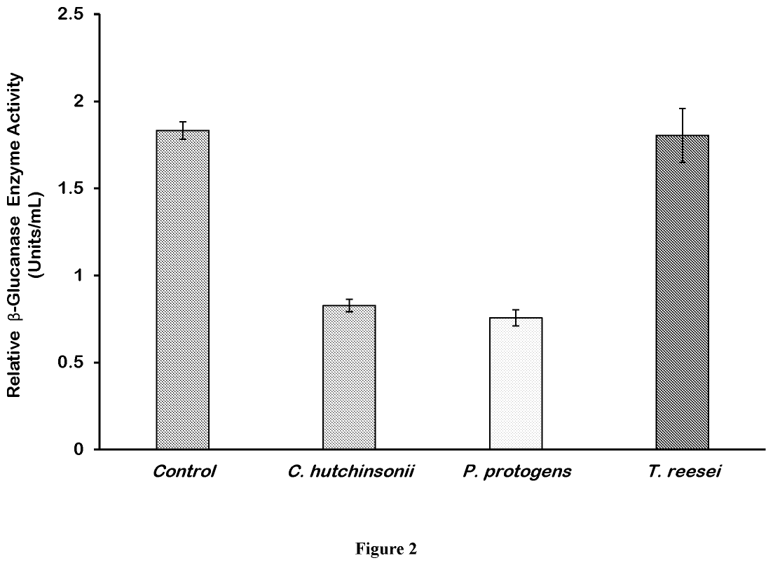

In referring to FIG. 2, one can observe a comparative graphical representation of the maximum β-glucanase enzymatic activity of differing microorganisms on MCA cellulose, which was obtained from the process described hereinabove. All bacteria and fungi tested demonstrated some enzyme activity, demonstrating the conversion of cellulose to cellobiose is occurring. C. hutchinsonii and P. protegens obtained a maximum enzyme activity of less than half of the control microorganism tested within the same amount of time, while T. reesei obtained the same level of activity as the control, but 7 days earlier (see Table 1).

TABLE 1
β-glucanase activity of microorganisms grown in the
presence of MCA cellulose for various incubation times

| | β-glucanase | Time to Maximum |
|---|---|---|
| Microorganism | Activity (Units/mL) | Activity (Days) |
| Control | 1.83 | 21 |
| C. hutchinsonii | 0.83 | 20 |
| P. protegens | 0.76 | 21 |
| T. reesei | 1.80 | 14 |

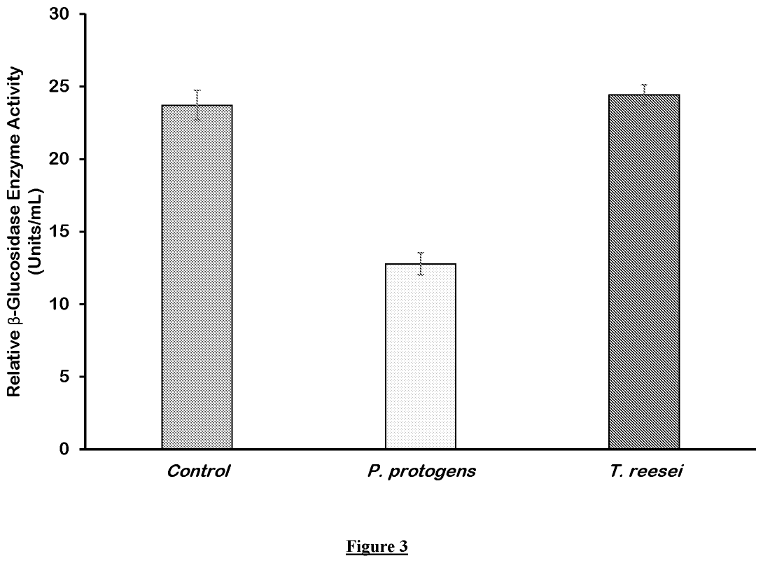

FIG. 3 illustrates the maximum β-glucosidase enzymatic activity of differing microorganisms incubated with MCA cellulose, which was obtained from a process as described hereinabove, as well as cellobiose. While all the microorganisms listed in Table 1 were tested, only P. protegens and T. reesei, in addition to the control microorganism, displayed enzymatic activity towards cellobiose, with T. reesei demonstrating the same amount of activity as the control microorganism in the same amount of incubation time (Table 2).

TABLE 2
β-glucosidase activity of microorganisms grown in the presence
of MCA cellulose and cellobiose for various incubation times

| | β-glucanase | Time to Maximum |
|---|---|---|
| Microorganism | Activity (Units/mL) | Activity (Days) |
| Control | 23.71 | 14 |
| P. protegens | 12.78 | 21 |
| T. reesei | 24.43 | 14 |

According to a preferred embodiment of the process of the present invention, the step of exposing a microorganism containing cellulase enzymes to MCA cellulose generates enzymatic activity, thus conversion of the cellulose to cellobiose and eventually, glucose. Cellulose, obtained through the delignification of a biomass feedstock by using a modified Caro's acid (such as MCA cellulose), has been shown to be biodegradable by several different bacteria and fungi. As is known, higher kappa numbers represent larger quantities of lignin which make a cellulosic material much more difficult to biodegrade. According to a preferred embodiment of the process of the present invention, this delignification process will enable a higher conversion of cellulose to glucose by overcoming the first step in the chain of reactions which is the conversion of cellulose to cellobiose by increasing the bioavailability of the cellulose to the microorganism or enzyme being utilized.

It is known to those skilled in the art that cellulose obtained from various Kraft processes has a lignin content of 2.5%-4.5% and a hemicellulose content of 9%-25%. MCA cellulose generated from the herein described delignification process results in a cellulose with less than 1% lignin content and a hemicellulose content of less than 15%.

It will be known by those skilled in the art that the process described herein provides significant benefits in comparison with existing state-of-the-art biomass delignification processes as it requires less energy due to the ambient conditions employed. In addition, the high delignification yields render the subsequent cellulose hydrolysis and fermentation highly efficient as the presence of lignin is known to be detrimental in currently existing processes due to residues in equipment and enzyme adsorption and deactivation. As a consequence to the lack of lignin, the resulting solids mostly comprising cellulose have a significantly higher surface area available to be degraded by enzymes and/or organisms, making this process highly efficient in terms of yield (of both monomeric and oligomeric sugars as well as fermentation products) and more cost-effective.

Given this information, it is believed that idle ethanol plants located around the world could re-start operations of cellulose conversion to glucose (and subsequently, ethanol) if a biomass feedstock according to the following specifications was employed rather than using corn, sugar cane or conventional kraft pulp. Moreover, the implementation of a process according to a preferred embodiment of the present invention would essentially “dovetail” with the delignification process of a lignocellulosic biomass by using a modified Caro's acid, and the production of ethanol with the cellulose obtained from the delignification process. As mentioned previously, the person skilled in the art will recognize that by employing a cellulose obtained from a process using a modified Caro's acid, one will circumvent the need of any further or subsequent bleaching step following the delignification. It is to be understood that the bleaching refers to a separate and distinct step of pulp processing. Consequently, the pulp used obtained using a modified Caro's acid driven delignification process, is intended on being a pulp which has not undergone a separate bleaching step post-delignification. As is also understood by the person skilled in the art, such a treatment step (bleaching) is understood to not be economically viable when the ultimate goal of the cellulose is to be further converted in order to generate ethanol. It is also understood by a person skilled in the art that such a high purity, low kappa number cellulose will be beneficial for cellulosic ethanol processes as it minimizes the issues brought by the presence of lignin in Kraft pulp processes or unbleached cellulose. It is known to those skilled in the art that lignin causes issues during the treatment of the cellulose portion as well as during the distillation of the hydrolysate. By utilizing a low kappa number cellulose obtained from a process using a modified Caro's acid, one will circumvent those issues, which will lead to increased bioethanol yields.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process to hydrolyze cellulose into cellobiose, said process comprising the following steps:
providing a reaction vessel;
providing a source of cellulose into said reaction vessel; wherein said source of cellulose having has a kappa number of less than 10, preferably the kappa number is 5 or less, more preferably the kappa number is 2 or less; and a hemicellulose content of less than 15%; preferably less than 10%, even more preferably less than 5% w/w of the total weight of the source of cellulose;
providing an organism capable of expressing one or more β-glucanases inoculum into said reaction vessel;
exposing said an organism capable of expressing one or more β-glucanases to said source of cellulose in an aqueous medium; and
optionally, recovering the supernatant comprising cellobiose.

2. The process according to claim 1 further comprising a step of exposing the cell supernatant to an organism to convert cellobiose to glucose, wherein said organism selected from the group consisting of: a bacterium, a fungus, a yeast, and a combination thereof, such as Aspergillus brasiliensis, Trichoderma reesei and Pseudomonas protegens.

3. The process according to claim 1 in which said organism capable of expressing one or more β-glucanase is selected from the group consisting of bacteria and fungi capable of producing endo-β-glucanase enzymes or exo-β-glucanase enzymes.

4. The process according to claim 1 in which said organism capable of expressing one or more β-glucanase is a bacterium of the phylum Bacteroidetes.

5. The process according to claim 1 in which said organism capable of expressing one or more β-glucanase is Cytophaga hutchinsonii.

6. The process according to claim 1 in which said organism capable of expressing one or more β-glucanase is a bacterium of the phylum Proteobacteria.

7. The process according to claim 1 in which said organism capable of expressing one or more β-glucanases is Pseudomonas protegens.

8. The process according to claim 1 in which the resulting glucose is exposed to one of Zymomonas mobilis or yeast Saccharomyces cerevisiae in a step to convert the glucose to ethanol.

9. The process according to claim 1 in which the organism used to convert cellobiose to glucose is a bacteria or fungi capable of producing β-glucosidase enzymes.

10. The process according to claim 1 in which the organism used to convert cellobiose to glucose is a fungus of the phylum Ascomycota.

11. The process according to claim 1 in which the organism used to convert cellobiose to glucose is a bacterium of the phylum Proteobacteria.

12. The process according to claim 1 wherein said source of cellulose is exposed to an organism at a temperature between 20° C. to 40° C.

13. The process according to claim 1 wherein said organism is incubated with said source of cellulose for a period of time ranging from 1 to 30 days.

14. The process according to claim 1 wherein said aqueous medium has a pH of about 5.0 to 8.0.

15. A process to hydrolyze cellulose into glucose, said process comprising the following steps:
providing a reaction vessel;
providing a source of cellulose into said reaction vessel; wherein said source of cellulose having has a kappa number of less than 10, preferably the kappa number is 5 or less, more preferably the kappa number is 2 or less; and a hemicellulose content of less than 15%; preferably less than 10% w/w; even more preferably less than 5% w/w of the total weight of the source of cellulose;
providing an inoculum of an organism capable of expressing one or more β-glucanases and β-glucosidase into said reaction vessel;
exposing said an organism capable of expressing one or more β-glucanases and β-glucosidase to said source of cellulose in an aqueous medium; and
optionally, recovering the supernatant comprising glucose.

16. The process according to claim 15 in which a bacteria or fungi capable of producing said organism capable of expressing endo- or exo-β-glucanase and β-glucosidase is used.

17. The process according to claim 15 wherein said bacteria or fungi capable of producing said organism capable of expressing endo- or exo-β-glucanase and β-glucosidase is selected from the group consisting of: Ascomycota, Proteobacteria, and combinations thereof.

18. The process according to claim 15 said an organism capable of expressing one or more β-glucanases and β-glucosidase is Trichoderma reesei.

19. The process according to claim 15 wherein said an organism capable of expressing one or more β-glucanases and β-glucosidase is Pseudomonas protegens.

20. The process according to claim 15 wherein said cellulose source is exposed to an organism at a temperature between 30° C. to 40° C.

21. The process according to claim 15 wherein said organism is incubated with said cellulose source for a period of time ranging from 1 to 30 days.

22. The process according to claim 15 wherein said aqueous medium has a pH of about 5.0 to 9.0.

23. Use of a source of cellulose having a kappa number of less than 10, preferably the kappa number is 5 or less, more preferably the kappa number is 2 or less; and a hemicellulose content of less than 15%; preferably less than 10% w/w, even more preferably, less than 5% w/w of the total weight of the source of cellulose; in a process to hydrolyze cellulose into cellobiose, wherein said process comprising the following steps:
providing a reaction vessel;
providing said source of cellulose into said reaction vessel;
providing an organism capable of expressing one or more β-glucanases into said reaction vessel;
exposing said an organism capable of expressing one or more β-glucanases to said source of cellulose in an aqueous medium; and
optionally, recovering the supernatant comprising cellobiose.

24. Use of a source of cellulose having a kappa number of less than 10, preferably the kappa number is 5 or less, more preferably the kappa number is 2 or less; and a hemicellulose content of less than 15%; preferably less than 10% w/w, even more preferably, less than 5% w/w of the total weight of the source of cellulose; in a process to hydrolyze cellulose into cellobiose (and optionally, to glucose or ethanol), wherein said process comprising the following steps:
providing a reaction vessel;
providing said source of cellulose into said reaction vessel;
providing an inoculum of an organism capable of expressing one or more β-glucanases and β-glucosidase into said reaction vessel;
exposing said an organism capable of expressing one or more β-glucanases and β-glucosidase to said source of cellulose in an aqueous medium;
optionally, recovering the supernatant comprising cellobiose;
optionally, exposing said supernatant comprising cellobiose to a bacteria or fungi that produces β-glucosidase for the conversion of cellobiose to glucose;
optionally, recovering the supernatant comprising glucose; and
optionally, exposing said supernatant comprising glucose to an ethanologenic bacteria or fungi for the fermentation glucose to ethanol.
